(19) United States
(12) Patent Application Publication
(10) Pub. No.: US 2005/0176619 A1
(43) Pub. Date: Aug. 11, 2005

(54) Antitumoral formulations of thioxanthenone

(76) Inventors: Ross Blundell, Jesmond (GB); Edward Baker, Chathill (GB)

Correspondence Address: ROSS J. OEHLER;AVENTIS PHARMACEUTICALS INC., ROUTE 202-206, MAIL CODE: D303A, BRIDGEWATER, NJ, 08807, US

(21) Appl. No.: 10/480,692

(22) PCT Filed: Jun. 28, 2002
(86) PCT No.: PCT/GB02/03012

(30) Foreign Application Priority Data
Jun. 28, 2001 (GB) 0115893.0

(51) Int. Cl. A61K 31/00

(52) U.S. Cl. 514/1

(57) ABSTRACT

The invention relates to a formulation of N-[[1-[[2-(diethylamino)ethyl]amino]-7-methoxy-9-oxothioxanthen-4-yl]-methyl]formamide, or a pharmaceutically acceptable acid-addition salt thereof, and to the method of use thereof in the treatment of tumors and cancers.

The invention relates to a formulation of N-[[1-[[2-(diethylamino)ethyl]amino]-7-methoxy-9-oxothioxanthen-4-yl]methyl]formamide, or a pharmaceutically acceptable acid-addition salt thereof, and to the method of use thereof in the treatment of tumors and cancers.

Brown et al., WO 97/10809, published Mar. 27, 2997, disclose reconstituted lyophilized formulations for the treatment of mammalian tumors comprising a thioxanthenone antitumor agent in combination with mannitol or sucrose as a stabilizer in a lactate buffer. The compound N-[[1-[[2-(diethylamino)ethyl]amino]-7-methoxy-9-oxothioxanthen-4-yl]methyl]formamide is specifically disclosed as example 31(a).

Brown et al., WO 97/11699, published Apr. 3, 1997, disclose aqueous parenteral formulations for the treatment of cancer tumors comprising 1,2,4-benzotriazine-3-amine 1,4-dioxides in a citrate buffer.

Stevenson et al., Cancer Chemother. Pharmacol. (1999), 44, pp. 228-234, disclose the phase I pharmacokinetic study results for the compound SR 233377 which was administered as a solution formulation containing 2.5 mg/mL of SR 233377 in an isotonic citrate buffer (pH 5.5).

LoRusso et al., Clinical Cancer Research, Vol. 6, 2000, pp. 3088-3094, disclose the phase I pharmacokinetic study results for the compound SR 233377 which was administered as a solution formulation containing 2.5 mg/mL of SR 233377 in an isotonic citrate buffer (H 5.5.).

Miller et al., U.S. Pat. No. 5,380,749, issued Jan. 10, 1995, disclose a series of 1-[[1-(dialkylamino)alkyl]amino]-4-substituted thioxanthen-9-one derivatives which are stated to be useful as antitumor agents. The compound N-[[1-[[2-(diethylamino)-ethyl]amino]-7-methoxy-9-oxothioxanthen-4-yl]methyl]formamide is specifically disclosed as example 31(a).

Perni et al., Journal of Medicinal Chemistry, 41(19), 1998, pp. 3645-3654, disclose the synthesis and antitumor activity of a series of 4-aminoethylthioxanthenone and 5-aminoethylbenzothiopyranoindazole derivatives. The compound N-[[1-[[2-(diethylamino)ethyl]amino]-7-methoxy-9-oxothioxanthen-4-yl]methyl]formamide is specifically disclosed as example 44.

It is desirable in the treatment of tumors and cancers to provide the active pharmaceutical ingredient as a ready-to-use (RTU) solution formulation which is pharmaceutically stable over prolonged periods of storage. Unfortunately, as N-[[1-[[2-(diethylamino)ethyl]amino]-7-methoxy-9-oxothioxanthen-4-yl]methyl]formamide is poorly soluble in pharmaceutically acceptable vehicles such as water (0.1 mg/mL) and is unstable under acidic conditions, formulating a ready-to-use solution formulation of this product is difficult. Accordingly, it is an object of the present invention to overcome these difficulties by providing a stable solution formulation of N-[[1-[[2-(diethylamino)ethyl]amino]-7-methoxy-9-oxothioxanthen-4-yl]methyl]formamide, or a pharmaceutically acceptable acid-addition salt thereof, in ready-to-use form.

More specifically, the invention relates to a formulation comprising N-[[1-[[2-(diethylamino)ethyl]amino]-7-methoxy-9-oxothioxanthen-4-yl]methyl]formamide, or a pharmaceutically acceptable acid-addition salt thereof; an acidic buffering agent; a pharmaceutically acceptable carrier, and a sufficient quantity of a base to adjust the pH of the formulation to from about 3 to about 8.

N-[[1-[[2-(diethylamino)ethyl]amino]-7-methoxy-9-oxothioxanthen-4-yl]methyl]formamide, which has the chemical structure shown below: 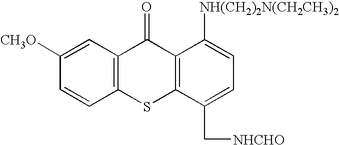
is a cytotoxic chemotherapeutic agent, as demonstrated by its antitumor/anticancer activity and, thus, is useful in the treatment of various types of susceptible cancers and tumors such as, for example, colon cancer, ovarian cancer, epidermoid cancer, cancers of germinal cells (e.g., testicular, mediastina, pineal gland), non-small cell lung cancers, non-Hodgkin's lymphoma, Hodgkin's disease, breast cancer, cancers of the upper respiratory and digestive tracts, gastric cancer, malignant melanoma, hepatocarcinoma, urothelial cancers, prostate cancers, small cell lung cancer, pancreatic cancer, gall bladder cancer, anal cancer, rectal cancer, bladder cancer, small intestine cancer, stomach cancer, leukemia and various other types of solid tumors or malignancies.

The preparation, physical properties and beneficial pharmacological properties of N-[[1-[[2-(diethylamino)ethyl]amino]-7-methoxy-9-oxothioxanthen-4-yl]methyl]formamide, or a pharmaceutically acceptable acid-addition salt thereof, are described in, for example, U.S. Pat. No. 5,380,749, International Application No. WO 97/10809, and Journal of Medicinal Chemistry, 41(19), 1998, pp. 3645-3654, the entire contents of each of which are herein incorporated by reference.

N-[[1-[[2-(diethylamino)ethyl]amino]-7-methoxy-9-oxothioxanthen-4-yl]methyl]formamide, or a pharmaceutically acceptable acid-addition salt thereof, is conveniently present in the formulations of the present invention in an amount of from about 0.1 mg/mL to about 100 mg/mL, preferably in the amount of from about 1 mg/mL to about 50 mg/mL, more preferably in the amount of from about 5 mg/ml to about 20 mg/mL, and in particular in the amount of about 10 mg/mL.

The term acidic buffering agent as used herein means any acid capable of solubilizing N-[[1-[[2-(diethylamino)ethyl]amino]-7-methoxy-9-oxothioxanthen-4-yl]methyl]formamide, or a pharmaceutically acceptable acid-addition salt thereof. Preferred acids are tricarboxylic acids, such as, for example, citric acid; dicarboxylic acids such as, for example, tartaric acid, maleic acid, succinic acid and fumaric acid; monocarboxylic acids such as, for example, acetic acid or lactic acid; or mineral acids such as, for example, hydrochloric acid and phosphoric acid; with the tricarboxylic acid citric acid being the most preferred.

The buffering agent is conveniently present in a molar concentration in the range of from about 0.01 M to about 0.1 M, most preferably in a molar concentration of about 0.02 M.

In the formulations of the present invention the ratio of N-[[1-[[2-(diethylamino)ethyl]amino]-7-methoxy-9-oxothioxanthen-4-yl]methyl]formamide, or a pharmaceutically acceptable acid-addition salt thereof, to the buffering agent is conveniently from about 1:1 to about 3:1, most preferably in the range of from about 2.4:1.

The term pharmaceutically acceptable carrier as used herein refers to the various solvents which can be employed in the preparation of the formulations of the present invention. In general, the carrier will be water, one or more other suitable solvents, or a mixture of water and one or more other suitable solvents. Preferably, the carrier is water. The water that is used is preferably pure water, i.e. sterile water for injection. Representative examples of the other suitable carriers (solvents) which can be utilized accordingly to the present invention include polyalkylene glycols, such as polyethylene glycol, polypropylene glycol, polybutylene glycol and the like and mixtures thereof; ethanol, propylene glycol; and glycerol.

The pH of the formulations of the present invention is generally in the range of about 3 to about 8, preferably in the range of about 4 to about 6, more preferably in the range of about 5 to about 5.5 and in particular about 5.2. The desired pH of the formulations of the present invention is obtained by adding a sufficient quantity of a base to the formulation. The base is preferably an alkali metal hydroxide or alkali metal citrate; more preferably an alkali metal hydroxide, and in particular sodium hydroxide. When sodium hydroxide is used as the base it is preferred to use about 0.01 M to 2.0 M of a solution of sodium hydroxide and in particular a 1.0 M solution of sodium hydroxide.

The formulations of the present invention may also optionally contain a tonicity modifier which, when used, is conveniently present in an amount from about 0.1% w/v to about 6% w/v, preferably in an amount from about 0.1% w/v to about 0.9% w/v and in particular in an amount of about 0.84% w/v. The tonicity modifier is preferably an alkali metal halide or dextrose, more preferably an alkali metal halide, and in particular sodium chloride.

The formulations of particular interest include those described in the accompanying examples and so formulations substantially as defined in the accompanying examples are provided as a further feature of the present invention.

A particularly preferred formulation of the present invention comprises 10 mg/mL of N-[[1-[[2-(diethylamino)ethyl]amino]-7-methoxy-9-oxothioxanthen-4-yl]methyl]formamide, water as the pharmaceutically acceptable carrier, 4.2 mg/mL of citric acid, and 8.4 mg/mL of sodium chloride, the pH of said formulation being adjusted to 5.2 with 1 M sodium hydroxide.

As mentioned above, N-[[1-[[2-(diethylamino)ethyl]amino]-7-methoxy-9-oxothioxanthen-4-yl]methyl]formamide, or a pharmaceutically acceptable acid-addition salt thereof, is a cytotoxic chemotherapeutic agent which is useful in the treatment of various types of susceptible cancers and tumors. Thus, the present invention also provides a method for the treatment of cancers or tumors in a mammal which comprises administering to a mammal in need of such treatment an effective amount of a formulation of the present invention.

The present invention further relates to the use of N-[[1-[[2-(diethylamino)ethyl]amino]-7-methoxy-9-oxothioxanthen-4-yl]methyl]formamide, or a pharmaceutically acceptable acid-addition salt thereof, in preparing a formulation according to the present invention for treating cancers or tumors in a mammal.

The present invention further relates to the use of the formulations of the invention in medicine.

The present invention further relates to a process for preparing the formulations of the invention which comprises mixing N-[[1-[[2-(diethylamino)ethyl]amino]-7-methoxy-9-oxothioxanthen-4-yl]methyl]formamide, or a pharmaceutically acceptable acid-addition salt thereof, an acidic buffering agent, a pharmaceutically acceptable carrier, a sufficient quantity of a base to adjust the pH of the formulation to from about 3 to about 8, and optionally a tonicity modifier.

The formulations of the present invention are generally administered to patients which include, but are not limited to, mammals, such as, for example, man, by conventional routes well known in the art For example, the formulations can be administered to patients orally, or parenterally (e.g., intravenously, intraperitoneally, and the like). The formulations are preferably administered parenterally, more preferably intravenously, and in particular by intravenous infusion. When infused intravenously, the formulation is generally diluted with a sodium chloride or dextrose solution prior to administration, preferably a 0.9% w/v sodium chloride solution, or a 5% w/v dextrose solution.

It will also be apparent to those skilled in the art that the formulations of the present invention can be administered with other therapeutic and/or prophylactic agents and/or medicaments that are not medically incompatible therewith.

The percentage of active component, i.e. N-[[1-[[2-(diethylamino)ethyl]amino]-7-methoxy-9-oxothioxanthen-4-yl]methyl]formamide, or a pharmaceutically acceptable acid-addition salt thereof, in the formulations of the present invention may be varied so that a suitable dosage is obtained. The dosage administered to a particular patient is variable depending upon the clinician's judgment using as criteria: the route of administration, the duration of treatment, the size, age and physical condition of the patient, the severity of the condition, the potency of the active component and the patient's response thereto. An effective dosage amount of the active component can thus readily be determined by the clinician after consideration of all criteria and using his best judgment on the patient's behalf. In general, the active component of the formulations of the present invention can be administered to patients in doses ranging from about 0.01 to about 100 mg/kg body weight.

The following examples will further illustrate the invention without, however, limiting it thereto. All temperatures are expressed in degrees Celsius (° C.).

The formulations of examples 1 to 14 set forth below were prepared by the following general procedure:

Dispense the acidic buffering agent, the tonicity modifier (if present), approximately 20% of the final weight of the pharmaceutically acceptable carrier and N-[[1-[[2-(diethylamino)ethyl]amino]-7-methoxy-9-oxothioxanthen-4-yl]methyl]formamide, or a pharmaceutically acceptable acid-addition salt thereof, into appropriate containers.

Charge the mixing vessel with a portion of the pharmaceutically acceptable carrier. Transfer the acidic buffering agent and the tonicity modifier (if present) from the containers to the mixing vessel and rinse the containers with a small amount of the pharmaceutically acceptable carrier. Mix the components in the vessel.

Transfer N-[[1-[[2-(diethylamino)ethyl]amino]-7-methoxy-9-oxothioxanthen-4-yl]-methyl]formamide, or a pharmaceutically acceptable acid-addition salt thereof, from the container to the mixing vessel and rinse the container with the remaining pharmaceutically acceptable carrier. Mix the components until all solids have dissolved to form a solution and measure the pH of the solution.

Prepare an appropriate solution of a base for pH adjustment.

Add additional pharmaceutically acceptable carrier to the mixing vessel to about 90% final weight of the solution. Mix the solution, and then measure the pH. Adjust the pH to the appropriate pH with the addition of a sufficient quantity of the base solution.

Add additional pharmaceutically acceptable carrier to make the solution up to the final weight. Mix the solution and then measure the final pH of the solution.

Transfer the solution to a pressure vessel under an inert atmosphere. Pressurize the vessel and then force the solution through a sterile 0.2 μm filter (e.g. a Millipore Duropore 47 mm filter which has a PVDF membrane) to sterilize the solution. Collect the filtered solution into a sterile glass holding vessel.

Fill the solution into suitable containers (e.g. vials, ampoules, flexible bags, syringes or bottles) with the headspace of the container being purged with an inert atmosphere before and after filling.

Immediately after purging the containers with an inert atmosphere, seal the containers.

It should be noted that while the above process is preferably carried out in the absence of an inert atmosphere, the formulations of the present invention can also be conveniently prepared under an inert atmosphere, such as nitrogen or argon, preferably nitrogen.

It should also be noted that while in the above process the formulation has been preferably sterilized by filtration, the formulations of the present invention can also be conveniently sterilized by heating in an autoclave.

TABLE 1

| | Ex. 1 | Ex. 2 | Ex. 3 | Ex. 4 | Ex. 5 | Ex. 6 | Ex. 7 | Ex. 8 | Ex. 9 | Ex. 10 | Ex. 11 | Ex. 12 |
|---|---|---|---|---|---|---|---|---|---|---|---|---|
| Active | 10 mg | 10 mg | 10 mg | 10 mg | 10 mg | 10 mg | 10 mg | 10 mg | 10 mg | 10 mg | 10 mg | 10 mg |
| ingredient* | | | | | | | | | | | | |
| Citric acid | 4.2 mg | 4.2 mg | 4.2 mg | 4.2 mg | 21 mg | 21 mg | 21 mg | 21 mg | 4.2 mg | 4.2 mg | 4.2 mg | 4.2 mg |
| monohydrate | (0.02M) | (0.02M) | (0.02M) | (0.02M) | (0.1M) | (0.1M) | (0.1M) | (0.1M) | (0.02M) | (0.02M) | (0.02M) | (0.02M) |
| Dextrose | 48.3 mg | 48.3 mg | 48.3 mg | 48.4 mg | 10.8 mg | 10.8 mg | 10.8 mg | 10.8 mg | — | — | — | — |
| Sodium | — | — | — | — | — | — | — | — | 8.4 mg | 8.4 mg | 8.4 mg | 8.4 mg |
| chloride | | | | | | | | | | | | |
| 1 M Sodium | 5.2 | 5.2 | 5.2 | 5.2 | 5.2 | 5.2 | 5.2 | 5.2 | 5.2 | 5.2 | 5.2 | 5.2 |
| hydroxide to | | | | | | | | | | | | |
| adjust pH to | | | | | | | | | | | | |
| Water for | 1 mL | 1 mL | 1 mL | 1 mL | 1 mL | 1 mL | 1 mL | 1 mL | 1 mL | 1 mL | 1 mL | 1 mL |
| injection to | | | | | | | | | | | | |
| Formulation | Yes | Yes | No | No | Yes | Yes | No | No | Yes | Yes | No | No |
| prepared under | | | | | | | | | | | | |
| nitrogen | | | | | | | | | | | | |
| Autoclaved at | Yes | No | Yes | No | Yes | No | Yes | No | Yes | No | Yes | No |
| 121° C. | | | | | | | | | | | | |
| for 15 minutes | | | | | | | | | | | | |

*N-[[1-[[2-(diethylamino)ethyl]amino]-7-methoxy-9-oxothioxanthen-4-yl]-methyl]formamide

TABLE 2

| | Example 13 | Example 14 |
|---|---|---|
| Active ingredient* | 50 mg | 50 mg |
| Citric acid Monohydrate | 10.5 mg (0.05 M) | 10.5 mg (0.05 M) |
| Sodium chloride | 9.05% w/v | — |
| Dextrose | — | 5.7% w/v |
| 1 M sodium hydroxide to | 5.0 to 5.5 | 5.0 to 5.5 |
| adjust pH to | | |
| Water for injection | 1 mL | 1 mL |

*N-[[1-[[2-(diethylamino)ethyl]amino]-7-methoxy-9-oxothioxanthen-4-yl]-methyl]formamide

The formulations of examples 1-14 were stored for up to 15 weeks and the stability results of the study are summarized in Table 3.

TABLE 3

| | | Δ Total Chromatographic Impurities |
|---|---|---|
| Example No. | Time | (TCI) % w/w |
| 1 | initial | 0.34 |
| | 2 wks at 40° C. | 0.47 |
| 2 | initial | 0.12 |
| | 8 wks at 5° C. | −0.05 |
| | 12 wks at 5° C. | −0.02 |
| | 4 wks at 25° C. | 0.09 |
| | 8 wks at 25° C. | 0.13 |
| | 15 days at 40° C. | 0.24 |
| | 4 wks at 40° C. | 0.45 |
| | 8 wks at 40° C. | 0.82 |
| 3 | Initial | 1.49 |
| | 2 wks at 40° C. | 1.40 |
| 4 | Initial | 0.16 |
| | 2 wks at 40° C. | 0.26 |
| | 4 wks at 40° C. | 0.59 |
| | 8 wks at 40° C. | 1.03 |
| 5 | Initial | 0.37 |
| | 2 wks at 40° C. | 0.99 |
| 6 | Initial | −0.05 |
| | 2 wks at 40° C. | 0.47 |
| | 4 wks at 40° C. | 1.12 |
| 7 | Initial | 0.63 |
| | 2 wks at 40° C. | 1.29 |
| 8 | Initial | 0.04 |
| | 2 wks at 40° C. | 0.51 |
| | 4 wks at 40° C. | 1.09 |
| 9 | Initial | 0.52 |
| | 2 wks at 40° C. | 0.30 |
| 10 | Initial | 0.11 |
| | 8 wks at 5° C. | −0.08 |
| | 12 wks at 5° C. | 0.00 |
| | 4 wks at 25° C. | 0.05 |
| | 8 wks at 25° C. | 0.05 |
| | 15 wks at 25° C. | 0.2 |
| | 15 days at 40° C. | 0.12 |
| | 4 wks at 40° C. | 0.2 |
| | 8 wks at 40° C. | 0.36 |
| 11 | Initial | 0.98 |
| | 2 wks at 40° C. | 1.21 |
| 12 | Initial | 0.08 |
| | 2 wks at 40° C. | 0.21 |
| | 4 wks at 40° C. | 0.43 |
| | 8 wks at 40° C. | 0.64 |
| 13 | 7 days at room | 0.05 |
| | temp | |
| | 7 days at 5° C. | −0.02 |
| 14 | 7 days at room | 0.06 |
| | temp | |
| | 7 days at 5° C. | −0.02 |

Δ TCI = the difference between the formulation TCI % w/w and the drug substance TCI % w/w (1.85 for examples 1-12 and 1.57 for examples 13-14)

1. A formulation comprising N-[[1-[[2-(diethylamino)ethyl]amino]-7-methoxy-9-oxothioxanthen-4-yl]-methyl]formamide, or a pharmaceutically acceptable acid-addition salt thereof; an acidic buffering agent; a pharmaceutically acceptable carrier; and a sufficient quantity of a base to adjust the pH of the formulation to from about 3 to about 8.

2. A formulation according to claim 1 wherein the ratio of N-[[1-[[2-(diethylamino)ethyl]amino]-7-methoxy-9-oxothioxanthen-4-yl]-methyl]formamide, or a pharmaceutically acceptable acid-addition salt thereof, to buffering agent is from about 1:1 to about 3:1.

3. A formulation according to claim 2 wherein the ratio of N-[[1-[[2-(diethylamino)ethyl]amino]-7-methoxy-9-oxothioxanthen-4-yl]-methyl]formamide, or a pharmaceutically acceptable acid-addition salt thereof, to buffering agent is from about 2.4:1.

4. A formulation according to claim 3 wherein the pharmaceutically acceptable carrier is water and the acidic buffering agent is citric acid.

5. A formulation according to claim 4 wherein the base is sodium hydroxide.

6. A formulation according to claim 5 wherein a sufficient quantity of sodium hydroxide is added to adjust the pH of the formulation to from about 4 to 6.

7. A formulation according to claim 6 wherein a sufficient quantity of sodium hydroxide is added to adjust the pH of the formulation to from about 5 to 5.5.

8. A formulation according to claim 7 wherein a sufficient quantity of sodium hydroxide is added to adjust the pH of the formulation to about 5.2.

9. A formulation according to claim 1 comprising from about 0.1 mg/mL to about 100 mg/mL of N-[[1-[[2-(diethylamino)ethyl]amino]-7-methoxy-9-oxothioxanthen-4-yl]-methyl]formamide, or a pharmaceutically acceptable acid-addition salt thereof.

10. A formulation according to claim 9 comprising from about 1 mg/mL to about 50 mg/mL of N-[[1-[[2-(diethylamino)ethyl]amino]-7-methoxy-9-oxothioxanthen-4-yl]-methyl]formamide, or a pharmaceutically acceptable acid-addition salt thereof.

11. A formulation according to claim 10 comprising from about 5 mg/mL to about 20 mg/mL of N-[[1-[[2-(diethylamino)ethyl]amino]-7-methoxy-9-oxothioxanthen-4-yl]-methyl]formamide, or a pharmaceutically acceptable acid-addition salt thereof.

12. A formulation according to claim 11 comprising about 10 mg/mL of N-[[1-[[2-(diethylamino)ethyl]amino]-7-methoxy-9-oxothioxanthen-4-yl]-methyl]formamide, or a pharmaceutically acceptable acid-addition salt thereof.

13. A formulation according to claim 1 further comprising a tonicity modifier.

14. A formulation according to claim 13 wherein the tonicity modifier is sodium chloride or dextrose and is present in an amount from about 0.1% w/v to about 6% w/v.

15. A formulation according to claim 14 wherein the tonicity modifier is sodium chloride and it is present in an amount from about 1.0% w/v to about 0.9% w/v.

16. A formulation according to claim 15 wherein the sodium chloride is present in an amount of about 0.84% w/v.

17. A formulation according to claim 16 comprising 10 mg/mL of N-[[1-[[2-(diethylamino)ethyl]amino]-7-methoxy-9-oxothioxanthen-4-yl]-methyl]formamide; water as the pharmaceutically acceptable carrier; 4.2 mg/mL of citric acid; and 8.4 mg/mL of sodium chloride, the pH of said formulation being adjusted to 5.2 with sodium hydroxide.

18. (canceled)

19. A method for the treatment of cancer or a tumor which comprises administering to a patient in need of such treatment an effective amount of a formulation according to claim 1.

20. (canceled)

21. A method for the treatment of cancer or a tumor which comprises administering to a patient in need of such treatment an effective amount of formulation according to claim 2.

22. A method for the treatment of cancer or a tumor which comprises administering to a patient in need of such treatment an effective amount of formulation according to claim 3

23. A method for the treatment of cancer or a tumor which comprises administering to a patient in need of such treatment an effective amount of formulation according to claim 4.

24. A method for the treatment of cancer or a tumor which comprises administering to a patient in need of such treatment an effective amount of formulation according to claim 5.

25. A method for the treatment of cancer or a tumor which comprises administering to a patient in need of such treatment an effective amount of formulation according to claim 6.

26. A method for the treatment of cancer or a tumor which comprises administering to a patient in need of such treatment an effective amount of formulation according to claim 7.

27. A method for the treatment of cancer or a tumor which comprises administering to a patient in need of such treatment an effective amount of formulation according to claim 8.

28. A method for the treatment of cancer or a tumor which comprises administering to a patient in need of such treatment an effective amount of formulation according to claim 9.

29. A method for the treatment of cancer or a tumor which comprises administering to a patient in need of such treatment an effective amount of formulation according to claim 10.

30. A method for the treatment of cancer or a tumor which comprises administering to a patient in need of such treatment an effective amount of formulation according to claim 11.

31. A method for the treatment of cancer or a tumor which comprises administering to a patient in need of such treatment an effective amount of formulation according to claim 12.

32. A method for the treatment of cancer or a tumor which comprises administering to a patient in need of such treatment an effective amount of formulation according to claim 13.

33. A method for the treatment of cancer or a tumor which comprises administering to a patient in need of such treatment an effective amount of formulation according to claim 14.

34. A method for the treatment of cancer or a tumor which comprises administering to a patient in need of such treatment an effective amount of formulation according to claim 15.

35. A method for the treatment of cancer or a tumor which comprises administering to a patient in need of such treatment an effective amount of formulation according to claim 16.

36. A method for the treatment of cancer or a tumor which comprises administering to a patient in need of such treatment an effective amount of formulation according to claim 17.
